(19) United States
(12) Patent Application Publication
(10) Pub. No.: US 2006/0251705 A1
(43) Pub. Date: Nov. 9, 2006

(54) Delivery of tigecycline in the presence of heparin

(75) Inventors: Stephen A. Ludwig, Chestnut Ridge, NY (US); Maja Vencl-Joncic, Highland Mills, NY (US); Pooja Gandhi, Highland Mills, NY (US); Joseph Gross, Goshen, NY (US); Julianna Koczone, Suffern, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/418,999

(22) Filed: May 5, 2006

Related U.S. Application Data
(60) Provisional application No. 60/678,203, filed on May 6, 2005.

(51) Int. Cl. A61K 31/727; A61K 31/65; A61K 9/70

(52) U.S. Cl. 424/443; 514/56; 514/154

(57) ABSTRACT

The present disclosure is directed to combination therapies of tigecycline and heparin and methods of administration of tigecycline and heparin.

“This application claims priority from copending provisional application, application No. 60/678,203 filed May 6, 2005 the entire disclosure of which is hereby incorporated by reference”

### CO-ADMINISTRATION OF TIGECYCLINE AND HEPARIN

In one embodiment, the present disclosure is directed to combination therapies of tigecycline and heparin and methods of administration of tigecycline and heparin.

Tigecycline, (9-(t-butyl-glycylamido)-minocycline, TBA-MINO, (4S,4aS,5aR,12aS)-9-[2-(tert-butylamino)acetamido]-4,7-bis(dimethylamino)-1,4,4a,5,5a,6,11,12a-octahydro-3,10,12,12a-tetrahydroxy-1,11-dioxo-2-naphthacenecarboxamide, is a glycylcycline antibiotic and an analog of the semisynthetic tetracycline, minocycline. Tigecycline is a 9-t-butylglycylamido derivative of minocycline, formula (I):

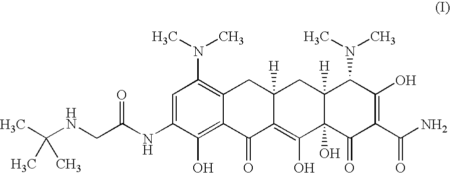

Tigecycline, developed in response to the worldwide threat of emerging resistance to antibiotics,has expanded broad-spectrum antibacterial activity both in vitro and in vivo. Glycylcycline antibiotics, like tetracycline antibiotics, act by inhibiting protein translation in bacteria.

Tigecycline is active against many antibiotic-resistant gram-positive pathogenic bacteria, such as methicillin-resistant Staphylococcus aureus, penicillin-resistant Streptococcus pneumoniae, and vancomycin-resistant enterococci (Eliopoulos, G. M. et al. 1994. Antimicrob Agents Chemother 38:534-41; Fraise, A. P. et al. 1995. Journal of Antimicrobial Chemotherapy. 35:877-81. [erratum appears in J Antimicrob Chemother May 1996;37(5):1046]; Garrison, M. W. et al. 2005. Clinical Therapeutics 27:12-22; Goldstein, F. W. et al. 1994. Antimicrobial Agents & Chemotherapy. 38:2218-20; Postier, R. G. et al. 2004. Clin Ther 26:704-714; Weiss, W. J. et al. 1995. Journal of Antimicrobial Chemotherapy. 36:225-30. Tigecycline is also active against bacterial strains carrying the two major forms of tetracycline resistance, efflux and ribosomal protection (Hirata, T. et al. 2004. Antimicrob Agents Chemother. 48:2179-84; Orth, P. et al. 1999. Journal of Molecular Biology 285:455-61; Projan, S. J. 2000. Pharmacotherapy 20:219S-223S; discussion 224S-228S; Schnappinger, D., and W. Hillen. 1996. Archives of Microbiology 165:359-69; Someya, Y. et al. 1995. Antimicrob Agents Chemother. 39:247-249.)

Tigecycline may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related bacteremia, Neutropenia, obstetrics and gynecological infections, or to treat other resistant pathogens, such as VRE, ESBL, enterics, rapid growing mycobacteria, and the like.

Hlavaka, et al., U.S. Pat. No. 5,529,990 discloses a method of treating or controlling bacterial infections in warm-blooded animals comprising administering a pharmacologically effective amount of a 7-substituted-9-(substituted amino)-6-demethyl-6-deoxytetracycline, of which tigecycline is a member of the genus described. U.S. Pat. No. 5,529,990 also discloses a method of treating or controlling bacterial infections in warm-blooded animals caused by bacteria having the TetM and TetK resistant determinants comprising administering a pharmacologically effective amount of a 7-substituted-9-(substituted amino)-6-demethyl-6-deoxytetracycline, of which tigecycline is a member of the genus described. U.S. Pat. No. 5,529,990 is incorporated herein by reference in its entirety.

Tigecycline may be manufactured by lyophilization and formulated, for example, compounded in the hospital pharmacy, for reconstitution as an IV solution. Tigecycline will frequently be administered simultaneously with other diluents and drugs. Since the tigecycline and the other diluents or drugs are often contained in separate infusion “bags”, the Y-sites on the administration set allow for the two solutions to be mixed together prior to using a common intravenous access point on the patient. Thus, in one embodiment, the tigecycline should be compatible with the other diluents or drugs when the two solutions are mixed together.

Heparin (“heparin”), an anticoagulant, is a heterogeneous group of straight-chain anionic mucopolysaccharides, called, glycosaminoglycans having anticoagulant properties. Injectable heparin sodium, for example, is indicated for anticoagulant therapy in prophylaxis and treatment of venous thrombosis and its extension; in a low-dose regimen for prevention of postoperative deep venous thrombosis and pulmonary embolism in patients undergoing major abdomino-thoracic surgery or who for other reasons are at risk of developing thromboembolic disease; prophylaxis and treatment of pulmonary embolism; atrial fibrillation with embolization; diagnosis and treatment of acute and chronic consumption coagulopathies (disseminated intravascular coagulation); prevention of clotting in arterial and heart surgery; prophylaxis and treatment of peripheral arterial embolism; and as an anticoagulant in blood transfusions, extracorporeal circulation, and dialysis procedures and in blood samples for laboratory purposes.

The tetracycline class (e.g., tetracycline, chlortetracycline, demeclocycline, minocycline, oxytetracycline, methacycline, and doxycycline) is a widely used antibiotic class. Some antibiotics in this class are known to be incompatible with the administration of heparin. Misgen, R., American Journal of Hospital Pharmacy (1965), 22(2), 92-4; Monnier, H. et al. ASHP Midyear Clinical Meeting, (December 1990) Vol. 25, p. P-186E. This incompatibility results in drawbacks in the administration of tetracyclines and heparin, such as, administration at the same site of a patient.

For example, minocycline, of which tigecylcine is an analog, has a demonstrated incompatibility with heparin. See Minocin Prescribing Information, p. 14, http://www.wyeth.com/content/ShowLabeling.asp?id=118, revised May 5, 2005, (stating that “Minocin IV should not be mixed before or during administration with any solutions containing . . . heparin sodium . . . ”)

The incompatibility between heparin and some previously known tetracyclines and tetracycline derivatives is often manifested as a visual incompatibility indicated by precipitation of solids. For example, when some tetracyclines and heparin chloride are administered on the same administration set, a precipitation is observed. This incompatibility usually requires a saline or other flush of the equipment prior to or after a heparin dose or requires a separate site of administration to the patient.

It has suprisingly been found here, however, that tigecycline may be administered with heparin. For example, tigecycline and heparin may be administered through a single administration site, such as through a Y-site mixing site, without a saline flush. In one embodiment, tigecycline and heparin do not have at least one of the incompatibilities of tetracyclines or tetracycline derivatives and heparin.

Disclosed is a composition comprising at least one glycylcycline, such as tigecycline and heparin. Another embodiment is a combination therapy comprising administration of at least one glycylcycline and heparin. A further embodiment is the coadministration of at least one glycylcycline and heparin. Another embodiment is a pharmaceutical composition comprising at least one glycylcycline and heparin and at least one pharmaceutically acceptable excipient. In one embodiment, tigecycline, as used herein, may be replaced or combined with other glycylcylcines. Also disclosed are methods of using at least one glycylcycline and heparin.

Another embodiment of the disclosure is a medical apparatus comprising at least two separate compartments, wherein a first compartment comprises at least one glycylcycline and a second compartment comprises heparin, and wherein the first and second compartments are connected by a line. For example, the first and second compartments may be connected to the same administration set and the contents of the first and second compartments may be mixed prior to administration. By further example, the administration set may contain a Y-site where the contents of the first and second compartments are mixed prior to administration.

### BRIEF DESCRIPTION OF THE DRAWINGS

FIG. 1 illustrates a common, clinical situation, where the admixed tigecycline is in the “Secondary” IV compartment, and the other diluents or drugs are in the “Primary” IV compartment.

### DEFINITIONS

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

“Glycylcycline” as used herein refers to any glycyl derivative of any tetracycline and includes any salt forms, such as any pharmaceutically acceptable salt, enantiomers and stereoisomers. See Sum P. E. et al. J Med Chem 1993;37:184-188. Glycylcycline, as used herein, may be formulated according to methods known in the art.

“Tigecycline” as used herein includes tigecycline in free base form and salt forms, such as any pharmaceutically acceptable salt, enantiomers and stereoisomers. Tigecycline, as used herein, may be formulated according to methods known in the art. By way of non-limiting example, tigecycline may be optionally combined with one or more pharmaceutically acceptable excipients, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight. Other formulations are discussed in U.S. Pat. Nos. 5,494,903 and 5,529,990, which are herein incorporated by reference.

“Heparin” as used herein includes heparin and its derivatives, including low and ultra-low molecular weight heparins and pharmaceutically acceptable salts, such as chlorine and sodium salts. Heparin may also be formulated according to methods known in the art.

“Pharmaceutical composition” as used herein refers to a medicinal composition.

“Pharmaceutically acceptable excipient” as used herein refers to pharmaceutical carriers or vehicles suitable for administration of the compounds provided herein including any such carriers known to those skilled in the art to be suitable for the particular mode of administration. For example, solutions or suspensions used for parenteral, intradermal, subcutaneous, or topical application can include a sterile diluent (e.g., water for injection, saline solution, fixed oil, and the like); a naturally occurring vegetable oil (e.g., sesame oil, coconut oil, peanut oil, cottonseed oil, and the like); a synthetic fatty vehicle (e.g., ethyl oleate, polyethylene glycol, glycerine, propylene glycol, and the like, including other synthetic solvents); antimicrobial agents (e.g., benzyl alcohol, methyl parabens, and the like); antioxidants (e.g., ascorbic acid, sodium bisulfite, and the like); chelating agents (e.g., ethylenediaminetetraacetic acid (EDTA) and the like); buffers (e.g., acetates, citrates, phosphates, and the like); and/or agents for the adjustment of tonicity (e.g., sodium chloride, dextrose, and the like); or mixtures thereof. By further example, where administered intravenously, suitable carriers include physiological saline, phosphate buffered saline (PBS), and solutions containing thickening and solubilizing agents such as glucose, polyethylene glycol, polypropyleneglycol, and the like, and mixtures thereof.

“Administration set” as used herein refers to a device used to administer fluids from a compartment to a patient's vascular system through a needle or catheter inserted into a vein. The device may include a needle or catheter, port(s) for administration set, roller clams, slide clamps, “primary” and “secondary” IV fluid compartments or containers, Y-injection sites, adapters, sample collection container or venous access site on a patient, tubing, flow regulators, drip chambers, in-line filters, IV set stopcocks, fluid delivery tubing, infusion pump, connectors between parts of the set, side tube with a cap to serve as an injection site, hollow spikes to penetrate and connect the tubing to IV bags or other infusion fluid compartments.

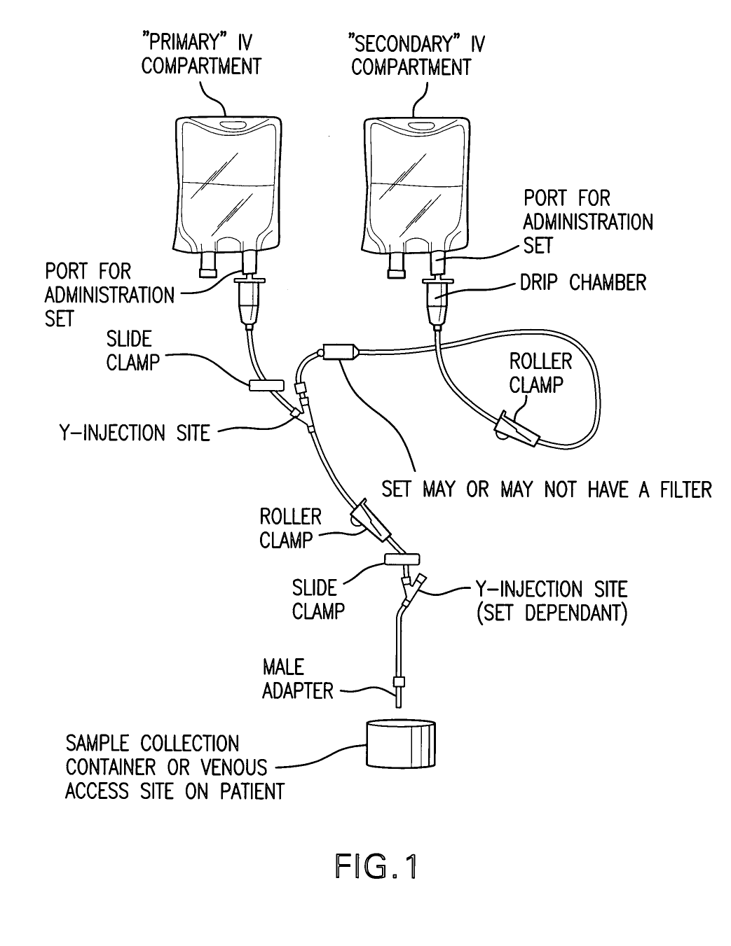

“Y-site” as used herein refers to a mixing site of an administration set. A non-limiting example is shown in FIG. 1.

“Incompatibility” as used herein refers to unsuitability for use of two drugs or diluents together because of chemical or other physical interaction.

“Co-administration” as used herein refers to administration of drug A at the same time as drug B, prior to or following the administration of drug B. In one embodiment, the administration is immediately prior or following. In an embodiment of the invention drug A is tigecycline and drug B is heparin sodium.

“Combination therapy” as used herein refers to a therapy that utilizes co-administration of drug A and drug B. In an embodiment of the invention drug A is tigecycline and drug B is heparin sodium.

“Administration” as used herein refers to providing a composition orally, parenterally (via intravenous injection (IV), intramuscular injection (IM), depo-IM, subcutaneous injection (SC or SQ), or depo-SQ), sublingually, intranasally (inhalation), intrathecally, topically, or rectally.

“Therapeutically effective amount” as used herein refers to an amount of a therapeutic agent administered to a host to treat or prevent a condition treatable by administration of a composition described in the invention. The amount is the amount sufficient to reduce or lessen at least one symptom of the disease being treated or to reduce or delay onset of one or more clinical markers or symptoms of the disease.

The terms “pharmaceutically acceptable salt” and “salts thereof” refer to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered. Pharmaceutically acceptable salts include salts of both inorganic and organic acids. Pharmaceutically acceptable salts include acid salts such as acetic, aspartic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methyinitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Other acceptable salts may be found, for example, in Stahl et al., Pharmaceutical Salts: Properties, Selection, and Use, Wiley-VCH; 1st edition (Jun. 15, 2002).

“Unit dosage form” used herein refers to physically discrete units suitable as unitary dosages, each unit containing a predetermined quantity of active material calculated to produce the desired therapeutic effect.

“Article of manufacture,” “medical apparatus,” and “medical product” as used herein refers to materials useful for prevention or treatment using, for example, tigecycline and heparin, such as a compartment with a label. The label can be associated with the article of manufacture in a variety of ways including, for example, the label may be on the compartment or the label may be in the compartment as a package insert. Suitable compartments include, for example, blister packs, bottles, bags, vials, syringes, test tubes, and the like. The compartments may be formed from a variety of materials such as glass, metal, plastic, rubber, paper, and the like. The article of manufacture may contain bulk quantities or less of tigecycline. The label on, or associated with, the compartment may provide instructions for the use of tigecycline, instructions for the dosage amount and for the methods of administration including compatibility with heparin. The article of manufacture may further comprise multiple compartments, also referred to herein as a kit. It may further include other materials desirable from a commercial and user standpoint, including other buffers, diluents, filters, needles, syringes, and/or package inserts with instructions for use.

“SWFI” is sterile water for injection.

“NS” is normal saline.

### DETAILED DESCRIPTION OF THE INVENTION

Tigecycline is an antibiotic that may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related bacteremia, Neutropenia, obstetrics and gynecological infections, or to treat other resistant pathogens, such as VRE, ESBL, enterics, rapid growing mycobacteria, and the like.

Other glycylcycline antibiotics may be used in place of tigecycline or in combination with tigecycline in the practice of the disclosure. Examples of other glycylcyclines include (9-(N,N-dimethylglycylamido)-6-demethyl-6-deoxytetracycline), (9-(N,N-dimethylglycylamido)-minocycline), and compounds included in U.S. Pat. No. 5,494,903, which is herein incorporated by reference.

In addition to tigecycline, patients may be receiving other diluents or drugs at the same site of administration. FIG. 1 illustrates a common, clinical situation, where the admixed tigecycline would be in the “Secondary” IV compartment, and the other diluents or drugs are in the “Primary” IV compartment. Mixing of the two fluids occurs at the Y-site, and the period that the two fluids are together is related to a number of variables (e.g. flow rates of each fluid, location of Y-site, and fluid volume in administration set from the Y-site to the venous access site).

In one embodiment, since there may be a common point of administration and thus mixing of tigecycline and one or more diluent or drug, the diluent or drug should be compatible with tigecycline.

It has been discovered that tigecycline is compatible with the administration of heparin, and, for example, can be administered to a patient at a common point of administration. In one embodiment, tigecycline and heparin may be administered through a single common point administration site, such as through a Y-site mixing point, without a saline flush. In one embodiment, tigecycline and heparin do not have at least one of the incompatibilities of tetracyclines or tetracycline derivates with heparin.

Disclosed is a composition comprising at least one glycylcycline chosen from a compound of the formula 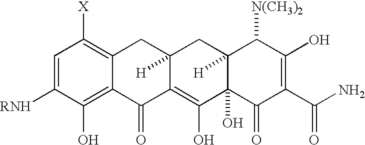
and pharmaceutically acceptable salts thereof, wherein:
X is selected from amino, NR¹R², or halogen; the halogen is selected from bromine, chlorine, fluorine or iodine; R¹ is selected from hydrogen, methyl, ethyl, n-propyl, 1-methylethyl, n-butyl and 1-methylpropyl; R² is selected from methyl, ethyl, n-propyl, 1-methylethyl, n-butyl, 1-methylpropyl, 2-methylpropyl, and 1,1-dimethylethyl such that when X═NR¹R² and R¹=hydrogen,
R²=methyl, ethyl, n-propyl, 1-methylethyl, n-butyl, 1-methylpropyl, 2-methylpropyl or 1,1-dimethylethyl; and when R¹=methyl or ethyl,
R²=methyl, ethyl, n-propyl, 1-methylethyl, n-butyl, 1-methylpropyl or 2-methylpropyl; and when R¹=n-propyl,
R²=n-propyl, 1-methylethyl, n-butyl, 1-methylpropyl or 2-methylpropyl; and when R¹=1-methylethyl,
R²=n-butyl, 1-methylpropyl or 2-methylpropyl; and when R¹=n-butyl,
R²=n-butyl, 1-methylpropyl or 2-methylpropyl; and when R¹=1-methylpropyl,
R²=2-methylpropyl;
R is selected from R⁴ (CH₂)ₙCO— or R^{4′} (CH₂)ₙSO2—; and n=0-4; and when R═R⁴ (CH₂)ₙCO— and n-0,
R⁴ is selected from amino; monosubstituted amino selected from straight or branched (C₁-C₆)alkylamino, cyclopropylamino, cyclobutylamino, benzylamino or phenylamino; disubstituted amino selected from dimethylamino, diethylamino, ethyl(1-methylethyl)amino, monomethylbenzylamino, piperidinyl, morpholinyl, 1-imidazolyl, 1-pyrrolyl, 1-(1,2,3-triazolyl) or 4-(1,2,4-triazolyl); a substituted (C₃-C₆)cycloalkyl group with substitution selected from cyano, amino or (C₁-C₃)acyl; a substituted (C₆-C₁₀)aryl group with substitution selected from halo, (C₁-C₄)alkoxy, trihalo (C₁-C₃)-alkyl, nitro, amino, cyano, (C₁-C₄)alkoxycarbonyl (C₁-C₃)alkylamino or carboxy; -amino-(C₁-C₄)alkyl selected from aminomethyl, .-aminoethyl, .-aminopropyl or .-amino-butyl; carboxy (C₂-C₄)-alkylamino selected from aminoacetic acid, .-aminobutyric acid or .-aminopropionic acid and the optical isomers thereof; (C₇-C₉)aralkylamino; (C₁-C₄)alkoxycarbonylamino substituted (C₁-C₄) alkyl group;
.-hydroxy(C₁-C₃)alkyl selected from hydroxymethyl, .-hydroxyethyl or .-hydroxy-1-methylethyl or .-hydroxypropyl; .-mercapto (C₁-C₃)alkyl selected from mercaptomethyl, .-mercaptoethyl, .-mercapto-1-methylethyl or .-mercaptopropyl; halo-(C₁-C₃)alkyl group; a heterocycle selected from the group consisting of a five membered aromatic or saturated ring with one N, O, S or Se heteroatom optionally having a benzo or pyrido ring fused thereto, a five membered aromatic ring with two N, O, S, or Se heteroatoms optionally having a benzo or pyrido ring fused thereto, a six membered aromatic ring with one to three N, O, S or Se heteroatoms, or a six membered saturated ring with one or two N, O, S or Se heteroatoms and an adjacent appended O heteroatom; acyl or haloacyl group selected from acetyl, propionyl, chloroacetyl, trifluoroacetyl; (C₃-C₆)cycloalcylcarbonyl, (C₆-C₁₀)aroyl selected from benzoyl or naphthoyl; halo substituted (C₆-C₁₀)aroyl; (C₁-C₄) alkylbenzoyl, or (heterocycle)-carbonyl, the heterocycle as defined hereinabove;
(C₁-C₄)alkoxycarbonyl selected from methoxycarbonyl, ethoxycarbonyl, straight or branched propoxylcarbonyl, straight or branched butoxycarbonyl or allyloxycarbonyl; a substituted vinyl group with substitution selected from halogen, halo(C₁-C₃)alkyl, or a substituted (C₆-C₁₀)aryl group with substitution selected from halo, (C₁-C₄)-alkoxy, trihalo(C₁-C₃)alkyl, nitro, amino, cyano, (C₁-C₄)alkoxycarbonyl, (C₁-C₃)alkylamino or carboxy;
(C₁-C₄)alkoxy group; C₆-aryloxy selected from phenoxy or substituted phenoxy with substitution selected from halo, (C₁-C₄) alkyl, nitro, cyano, thiol, amino, carboxy, di(C₁-C₃)alkylamino; (C₇-C₁₀)aralkyloxy; vinyloxy or a substituted vinyloxy group with substitution selected from (C₁-C₄)alkyl, cyano, carboxy, or (C₆-C₁₀)aryl selected from phenyl, .-naphthyl or .-naphthyl; R^{a}R^{b} amino(C₁-C₄)alkoxy group, wherein R^{a}R^{b} is a straight or branched (C₁-C₄)alkyl selected from methyl, ethyl, n-propyl, 1-methylethyl, n-butyl, 1-methylpropyl, or 2-methylpropyl or R^{a}R^{b} is (CH2)m, m=2-6, or (CH₂)₂ W(CH₂)₂—wherein W is selected from —N(C₁-C₃)alkyl,O,S, —NH, —NOB and B is selected from hydrogen or (C₁-C₃)alkyl; or R^{a}R^{b} aminoxy group, wherein R^{a}R^{b} is a straight or branched (C₁-C₄)alkyl selected from methyl, ethyl, n-propyl, 1-methylethyl, n-butyl, 1-methylpropyl, 2-methylpropyl, or 1,1-dimethylethyl or R^{a}R^{b} is (CH2)m, m=2-6, or —(CH₂)₂ W(CH₂)₂— wherein W is selected from —N(C₁-C₃)alkyl, O,S, —NH, —NOB and B is selected from hydrogen or (C₁-C₃)alkyl;
and when R═R⁴ (CH₂)ₙ CO— and n-1-4, R⁴ is selected from amino;
a substituted (C₃-C₆)cycloalkyl group with substitution selected from cyano, amino or (C₁-C₃)acyl; a substituted(C₆-C₁₀)-aryl group with substitution selected from halo, (C₁-C₄)-alkoxy, trihalo(C₁-C₃)alkyl, nitro, amino, cyano,(C₁-C₄)alkoxycarbonyl, (C₁-C₃)alkylamino or carboxy; acyloxy or haloacyloxy group selected from acetyl, propionyl, chloroacetyl, trichlorocetyl, (C₃-C₆)cycloalkylcarbonyl, (C₆-C₁₀)aroyl selected from benzoyl or naphthoyl, halo substituted (C₆-C₁₀)aroyl, (C₁-C₄)alkylbenzoyl, or (heterocycle)-carbonyl, the heterocycle as defined hereinabove;
(C₁-C₄)alkoxy; C₆-aryloxy selected from phenoxy or substituted phenoxy with substitution selected from halo, (C₁-C₄)-alkyl, nitro, cyano, thiol, amino, carboxy, di(C₁-C₃)-alkylamino; (C₇-C₁₀)aralkyloxy; (C₁-C₃)alkylthio group selected from methylthio, ethylthio, propylthio or allythio; C₆-arylthio group selected from phenylthio or substituted phenylthio with substitution selected from halo, (C₁-C₄)alkyl, nitro, cyano, thiol, amino, carboxy, di(C₁-C₃)alkylamino; C₆-arylsulfonyl group selected from phenylsulfonyl or substituted phenylsulfonyl with substitution selected from halo, (C₁-C₄)alkoxy, trihalo(C₁-C₃)alkyl, nitro, amino, cyano, (C₁-C₄)alkoxycarbonyl, (C₁-C₃)alkylamino or carboxy; (C7-C8)aralkylthio group; a heterocycle as defined hereinabove; hydroxy; mercapto; mono- or di-straight or branched chain (C₁-C₆)-alkylamino with the alkyl selected from methyl, ethyl, n-propyl, 1-methylethyl, n-butyl, 1-methylpropyl, 2-methylpropyl, 1,1-dimethylethyl, 2-methylbutyl, 1,1-dimethylpropyl, 2,2-dimethylpropyl, 3-methylbutyl, n-hexyl, 1-methylpentyl, 1,1-dimethylbutyl, 2,2-dimethylbutyl, 2-methylpentyl, 1,2-dimethylbutyl, 1,3-dimethylbutyl or 1-methyl-1-ethylpropyl; (C₂-C₅)azacycloalkyl group; a carboxy(C₂-C₄)alkylamino group with the carboxy alkyl selected from aminoacetic acid, -aminopropionic acid, -aminobutyric acid and the optical isomers thereof; .-hydroxy(C₁-C₃)alkyl selected from hydroxymethyl, .-hydroxyethyl or .-hydroxy-1-methylethyl or .-hydroxypropyl; halo(C₁-C₃)alkyl group; acyl or haloacyl selected from acetyl, propionyl, chloroacetyl, trifluoroacetyl; (C₃-C₆)cycloalkylcarbonyl; (C₆-C₁₀)aroyl selected from benzoyl or naphthoyl; halo substituted (C₆-C₁₀)aroyl; (C₁-C₄)alkylbenzoyl, or (heterocycle)carbonyl, the heterocycle as defined hereinabove;
(C₁-C₄)alkoxycarbonylamino, group selected from tert-butoxycarbonylamino, allyloxycarbonylamino, methoxycarbonylamino, ethoxycarbonylamino or propoxycarbonylamino; (C₁-C₄)alkoxycarbonyl group selected from methoxycarbonyl, ethoxycarbonyl, straight or branched propoxycarbonyl, allyloxycarbonyl or straight or branched butoxycarbonyl; R^{a}R^{b}-amino(C₁-C₄)alkoxy group wherein R^{a}R^{b} is a straight or branched (C₁-C₄)alkyl selected from methyl, ethyl, n-propyl, 1-methylethyl, n-butyl, 1-methylpropyl, or 2-methylpropyl or R^{a}R^{b} is (CH)ₘ m=2-6 or —(CH₂)₂ W(CH₂)₂— wherein W is selected from —N(C₁-C₃)-alkyl, O, S, —NH, —NOB, and B is selected from hydrogen or C₁-C₃)alkyl; or R^{a}R^{b} aminoxy group, wherein R^{a}R^{b} is a straight or branched (C₁-C₄)-alkyl selected from methyl, ethyl, n-propyl, 1-methylethyl, n-butyl, 1-methylpropyl, 2-methylpropyl or R^{a}R^{b} is (CH2)ₘ, m=2-6, or —(CH₂)₂ W(CH₂)₂— wherein W is selected from —N(C₁-C₃)-alkyl, O,S, —NH, —NOB and B is selected from hydrogen or (C₁-C₃)alkyl, and when R═R⁴ (CH₂)ₙ SO₂— and n=0
R⁴ is selected from amino; monosubstituted amino selected from straight or branched (C₁-C₆)alkylamino, cyclopropylamino, cyclobutylamino, benzylamino or phenylamino; disubstituted amino selected from dimethylamino, diethylamino, ethyl(1-methylethyl)amino, monomethylbenzylamino, piperidinyl, morpholinyl, 1-imidazoyl, 1-pyrrolyl, 1-(1,2,3-triazolyl) or 4-(1,2,4-triazolyl); a substituted (C₃-C₆)cycloalkyl group with substitution selected from cyano, amino or (C₁-C₃)acyl; halo(C₁-C₃)alkyl group; a heterocycle as defined hereinabove;
R^{a}R^{b} amino (C₁-C₄) alkoxy group, wherein R^{a}R^{b} is a straight or branched (C₁-C₄)-alkyl selected from methyl, ethyl, n-propyl, 1-methyl-ethyl, n-butyl, 1-methylpropyl, or 2-methylpropyl or R^{a}R^{b} is (CH2)m, m=2-6, or —(CH₂)₂W—(CH₂)₂— wherein W is selected from —N(C₁-C₃) alkyl, O, S, —NH, —NOB and B is selected from hydrogen or (C₁-C₃)-alkyl; or R^{a}R^{b} aminoxy group, wherein R^{a}R^{b} is a straight or branched (C₁-C₄)alkyl selected from methyl, ethyl, n-propyl, 1-methylethyl, n-butyl, 1-methyl-propyl, or 2-methyl-propyl or R^{a}R^{b} is (CH2)m, m=2-6, or —(CH₂)₂W(CH₂)₂— wherein W is selected from —N(C₁-C₃) alkyl, O, S, —NY, —NOB and B is selected from hydrogen or (C₁-C₃) alkyl; and when R═R⁴ (CH₂)ₙSO₂— and n=1-4,
R^{4′} selected from C₁-C₄)carboxyalkyl; a substituted (C₃-C₆)cyclalkyl group with substitution selected from cyano, amino or (C₁-C₃)-acyl; (C₁-C₄)alkoxy; C₆-aryloxy selected from phenoxy or substituted phenoxy with substitution selected from halo, (C₁-C₃)alkyl, nitro, cyano, thiol, amino, carboxy, di(C₁-C₃) alkylamino; (C₇-C₁₀)aralkyloxy; R^{a}R^{b} amino (C₁-C₄) alkoxy, wherein R^{a}R^{b} is a straight or branched (C₁-C₄)-alkyl selected from methyl, ethyl, n-propyl, 1-methylethyl, n-butyl, 1-methylpropyl, or 2-methylpropyl or R^{a}R^{b} is (CH2)m, m=2-6, or —(CH₂)₂W(CH₂)₂— wherein W is selected from —N(C₁-C₃)alkyl, O,S, —NY, or NOB and B is selected from hydrogen or (C₁-C₃)alkyl; or R^{a}R^{b} aminoxy group, wherein R^{a}R^{b} is a straight or branched (C₁-C₄)alkyl selected from methyl, ethyl, n-propyl, 1-methylethyl, n-butyl, 1-methylpropyl, or 2-methylpropyl or R^{a}R^{b} is (CH2)ₘ, m=2-6, or —(CH₂)₂W(CH₂)₂— wherein W is selected from —N(C₁-C₃)alkyl, O,S, —NH, —NOB and B is selected from hydrogen or (C₁-C₃)alkyl; (C₁-C₃) alkylthio selected from methylthio, ethylthio or n-propylthio; C₆-arylthio selected from phenylthio or substituted phenylthio with substitution selected from halo, (C₁-C₃)alkyl, nitro, cyano, thiol, amino, carboxy, di(C₁-C₃)alkylamino; (C₇-C₈) aralkylthio; a heterocycle as defined hereinabove; hydroxy; mercapto; mono- or di-straight or branched (C₁-C₆)alkyl-amino group the alkyl selected from methyl, ethyl, n-propyl, 1-methylethyl, n-butyl, 1-methylpropyl, 2-methylpropyl, 1,1-dimethylethyl, 2-methylbutyl, 1,1-dimethylpropyl, 2,2-dimethylpropyl, 3-methylbutyl, n-hexyl, 1-methylpentyl, 1,1-dimethylbutyl, 2,2-dimethylbutyl, 2-methylpentyl, 1,2-dimethylbutyl, 1,3-dimethylbutyl or 1-methyl-1-ethylpropyl; halo (C₁-C₃) alkyl; acyl or haloacyl selected from acetyl, propionyl, chloro-acetyl, trifluoroacetyl; (C₃-C₆) cycloalkylcarbonyl; (C₆-C₁₀) aroyl selected from benzoyl or naphthoyl; halo substituted (C₆-C₁₀)aroyl, (C₁-C₄) alkylbenzoyl, or (heterocycle) carbonyl, the heterocycle as defined hereinabove; (C₁-C₄)alkoxycarbonyl selected from methoxycarbonyl, ethoxycarbonyl, straight or branched propoxycarbonyl, allyloxycarbonyl or straight or branched butoxycarbonyl; R⁵ is selected from hydrogen; straight or branched (C₁-C₃) alkyl selected from methyl, ethyl n-propyl or 1-methylethyl; (C₆-C₁₀)aryl selected from phenyl, .-naphthyl or .-naphthyl; (C₇-C₉) aralkyl group; a heterocycle as defined hereinabove; or —(CH₂)ₙCOOR⁷ where n=0-4 and R⁷ is selected from hydrogen; straight or branched (C₁-C₃)alkyl group selected from methyl, ethyl, n-propyl or 1-methylethyl; or (C₆-C₁₀)aryl group selected from phenyl, -naphthyl or .-naphthyl;
R⁶ is selected from hydrogen; straight or branched (C₁-C₃)alkyl group selected form methyl, ethyl, n-propyl or 1-methylethyl; (C₆-C₁₀)aryl group selected from phenyl, -naphthyl or .-naphthyl; (C₇-C₉)-aralkyl group; a heterocycle as defined hereinabove; or —CH₂)ₙ (COOR⁷ where n=0-4 and R^{7′} is selected from hydrogen; straight or branched (C₁-C₃)alkyl selected from methyl, ethyl, n-propyl or 1-methylethyl; or (C₆-C₁₀)aryl selected from phenyl, -naphthyl or .-naphthyl; with the proviso that R⁵ and R⁶ cannot both be hydrogen; or R⁵ and R⁶ taken together are —(CH₂)₂W(CH₂)₂—, wherein W is selected from (CH₂)_{q} and q=0-1, —NH, —N(C₁-C₃)-alkyl, —N(C₁-C₄) alkoxy, oxygen, sulfur or substituted congeners selected from (L or D) proline, ethyl (L or D) prolinate, morpholine, pyrrolidine or piperidine;
   or compounds included in U.S. Pat. No. 5,494,903 which is herein incorporated by reference and at least one heparin.

In one embodiment of the disclosure, the at least one glycylcycline is tigecycline. For example, a composition comprising at least one glycylcycline chosen from a compound of formula I 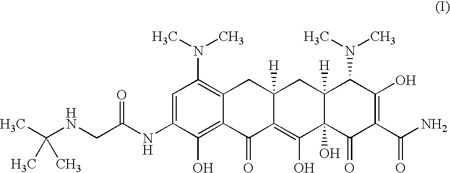
and pharmaceutically acceptable salts thereof, and at least one heparin. For example, the heparin may be heparin sodium. In another embodiment the composition is suitable for parenteral, specifically intravenous, administration.

Another embodiment is a pharmaceutical composition comprising at least one glycylcycline, at least one heparin and at least one pharmaceutically acceptable excipient. For example, the heparin may be heparin sodium. In another embodiment the composition is suitable for parenteral, specifically intravenous, administration.

Another embodiment is a combination therapy comprising administration of at least one glycylcycline and at least one heparin either sequentially or as a mixture. For example, the combination therapy may produce no incompatibility as determined by at least one test chosen from color change, gas formation, visible particulate formation, turbidity and sub visible particulate formation. For example, the heparin may be heparin sodium. In another embodiment the combination therapy is suitable for parenteral, specifically intravenous, administration. By further example, the at least one glycylcycline and the at least one heparin are administered at substantially the same time or are administered between two hours before and two hours after the at least one heparin is administered, such as for example 15 minutes, 30 minutes, 1 hour or 2 hours. For example, the heparin may be administered at the substantially the same time as tigecycline, with subsequent administration of tigecycline at, for example, 6, 12, 24 or 48 hour intervals thereafter for at least one dosing interval after initial administration. Further, for example, the heparin may be administered at substantially the same time as tigecycline, with subsequent administration of tigecycline at 12 hour intervals thereafter for 4 days after the initial administration.

Another embodiment of the disclosure is a medical apparatus comprising at least two separate compartments, wherein a first compartment comprises at least one glycylcycline and a second compartment comprises at least one heparin, and wherein the first and second compartments are connected to an administration set. For example, the first and second compartments may be connected to the same administration set and the contents of the first and second compartments may be mixed prior to administration. Optionally each compartment may be separate IV bags. By further example, the administration set may contain a Y-site where the contents of the first and second compartments are mixed prior to administration. In another embodiment, the flushing of the administration set or mixing point is not required.

Another embodiment is a method for administering at least one glycylcycline and at least one heparin, comprising administering to a patient in need thereof a therapeutically effective amount of the at least one glycylcycline, and administering to a patient in need thereof a therapeutically effective amount of at least one heparin. For example, the at least one glycylcycline and the heparin may be administered through the same site of administration. For example, this method and other methods and compositions disclosed may produce no incompatibility as determined by at least one test chosen from color change, gas formation, visible particulate formation, turbidity and sub visible particulate formation. For example, the heparin may be heparin sodium. In another embodiment the composition is suitable for parenteral, specifically intravenous, administration. By further example, the at least one glycylcycline and the at least one heparin are administered at substantially the same time or are administered between two hours before and two hours after the at least one heparin is administered, such as for example 15 minutes, 30 minutes, 1 hour or 2 hours. For example, the heparin may be administered at the substantially the same time as tigecycline, with subsequent administration of tigecycline at, for example, 6, 12, 24 or 48 hour intervals thereafter for at least one dosing interval after initial administration. Further, for example, the heparin may be administered at substantially the same time as tigecycline, with subsequent administration of tigecycline at 12 hour intervals thereafter for 4 days after the initial administration.

A further embodiment is a method of administering an antibiotic comprising administering to a patient in need thereof a therapeutically effective amount of at least one glycylcycline, and administering to a patient in need thereof a therapeutically effective amount of at least one heparin. For example, the glycylcycline and the heparin are administered through the same site of administration. By further example, the at least one glycylcycline and the at least one heparin are administered at substantially the same time or are administered between two hours before and two hours after the at least one heparin is administered, such as for example 15 minutes, 30 minutes, 1 hour or 2 hours. For example, the heparin may be administered at the substantially the same time as tigecycline, with subsequent administration of tigecycline at, for example, 6, 12, 24 or 48 hour intervals thereafter for at least one dosing interval after initial administration. Further, for example, the heparin may be administered at substantially the same time as tigecycline, with subsequent administration of tigecycline at 12 hour intervals thereafter for 4 days after the initial administration.

Another embodiment is a method of treating bacterial infections, such as complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) comprising administering to a patient in need thereof a therapeutically effective amount of at least one glycylcycline, and administering to a patient in need thereof a therapeutically effective amount of at least one heparin. By further example, in the methods disclosed, the at least one glycylcycline and the at least one heparin are administered at substantially the same time or are administered between two hours before and two hours after the at least one heparin is administered, such as for example 15 minutes, 30 minutes, 1 hour or 2 hours. For example, the heparin may be administered at the substantially the same time as tigecycline, with subsequent administration of tigecycline at, for example, 6, 12, 24 or 48 hour intervals thereafter for at least one dosing interval after initial administration. Further, for example, the heparin may be administered at substantially the same time as tigecycline, with subsequent administration of tigecycline at 12 hour intervals thereafter for 4 days after the initial administration.

Another embodiment is a method of administering an antibiotic to a patient receiving heparin comprising administering to a patient in need thereof a therapeutically effective amount of at least one glycylcycline. Another embodiment is a method of administering at least one glycylcycline to a patient receiving heparin comprising administering to a patient in need thereof a therapeutically effective amount of at least one glycylcycline.

Another embodiment is a method of using at least one glycylcycline in the treatment of a bacterial infection, such as those disclosed herein comprising providing a patient with a therapeutic amount of at least one glycylcycline and informing the patient and/or administering medical personnel that the at least one glycylcycline is compatible with heparin, for example the glycylcycline will not produce an incompatibility with heparin at a common point of administration.

Another embodiment is a composition comprising at least one glycylcycline, such as a pharmaceutical composition, or any composition disclosed herein comprising packaging with information that the at least one glycylcycline, such as tigecycline, may be administered with heparin. For example, the packaging may explain that there is no incompatibility at a common point of administration. Also disclosed in a method of supplying such a composition to medical personal or a patient in need thereof.

Another embodiment is a method or composition disclosed herein that further comprises a container or compartment with printed labeling advising that the at least one glycylcycline, such as tigecycline, may be administered with heparin. For example, advising that there is incompatibility at a common point of administration or that a composition disclosed herein can be administered with heparin, for example, at a common point of administration. For example, a composition or method disclosed here may further provide information that the administration of a therapeutically effective amount of at least one glycylcycline with heparin does not produce an incompatibility at a common point of administration.

One embodiment is packaging a composition comprising at least one glycylcycline with information at the at least one glycylcycline may be administered with heparin at a common point of administration without flushing the common point of administration, and methods of using such a composition.

In another embodiment, at least one glycylcycline may be provided in kits, optionally including component parts that can be assembled for use. For example, at least one glycylcycline in lyophilized form and a suitable diluent may be provided as separated components for combination prior to use. A kit may include a plurality of compartments, each compartment holding at least one unit dose of at least one glycylcycline. The compartments are preferably adapted for the desired mode of administration, including, for example, pill, tablet, capsule, powder, gel or gel capsule, sustained-release capsule, or elixir form, and/or combinations thereof, and the like for oral administration, depot products, pre-filled syringes, ampoules, vials, and the like for parenteral administration, and patches, medipads, creams, and the like for topical administration.

For example, a kit may comprise (a) at least one dosage form of at least one glycylcycline; (b) at least one compartment in which at least one glycylcycline is stored; and (c) a package insert comprising at least one of: i) information regarding the dosage amount and duration of exposure of a dosage form of at least one glycylcycline and ii) providing that the dosage form of at least one glycylcycline may be administered with heparin at a common point of administration.

In another embodiment, an article of manufacture may comprise a compartment holding at least one glycylcycline in combination with printed labeling instructions providing a discussion that indicates the compatibility of heparin and the at least one glycylcycline, for example, as opposed to other tetracyclines. The labeling instructions may be consistent, for example, with the methods of treatment as described herein before. The labeling may be associated with the compartment by any means that maintain a physical proximity of the two, by way of non-limiting example, they may both be contained in a packaging material such as a box or plastic shrink wrap or may be associated with the instructions being bonded to the compartment such as with glue that does not obscure the labeling instructions or other bonding or holding means.

For example, an article of manufacture may comprise(a) a dosage form of at least one glycylcycline, (b) a package insert or printed labeling providing that a dosage form of the at least one glycylcycline may be co-administered with heparin without flushing of a common administration site; and (c) at least one compartment in which the at least one glycylcycline is stored.

Compatibility of two or more compounds or compositions, such as tigecycline and heparin, are tested using at least one of color change, gas formation, visible particle formation, turbidity and sub-visible particle formation.

For example, color change, gas formation, visible particulate formation, and turbidity may be measured using a Black and White background light box with a fluorescent lamp capable of producing intensity of illumination between 2000 and 3750 lux.

Sub-visible particulate formation may be measured by light obscuration (HIAC) as per United States Pharmacopeia USP Chapter 788 Particulate Matter in Injections.

As a non-limiting example, an effective amount of tigecycline ranges from 0.5 mg/kg of body weight to 100.0 mg/kg of body weight, for example, from 0.5 to 15 mg/kg of body weight, by further example, from 0.5 to 1 mg/kg of body weight, may be administered from one to five times per day. For example, tigecycline may be administered as a 100 mg loading dose followed by subsequent administration of 50 mg both administered to the patient via infusion over a 30-60 minute period. In the above example, the loading dose of 100 mg may be prepared by adding two vials of reconstituted tigecycline to 100 mL Normal Saline or dextrose 5% in water (“D5W”) intravenous compartments resulting in a final concentration of 1 mg/mL, whereas the subsequent dose of 50 mg may be prepared by adding one vial of reconstituted tigecycline to 100 mL Normal Saline or D5W intravenous compartments resulting in a final concentration of 0.5 mg/mL. It will be understood, however, that the specific dose level and frequency of dosage for any particular patient may be varied and will depend on a variety of factors including age, body weight, general health, sex, diet, the severity of the condition being treated, and the like.

The reconstitution of tigecycline would be understood by one skilled in the art, for example, by following instructions included by the manufacturer or distributor or by using common medical procedures, which are inclusive of using only a sterile acceptable reconstitution medium and sterile administration compartments as described herein and in the tigecycline product label, to reconstitute and administer the lyophilized tigecycline free base supplied by the manufacturer.

Therapeutically affective amounts of heparin and formulations of heparin are well know in the art. For example, for continuous intravenous administration, an initial dose of 5,000 Units by IV injection followed by continuous dosing of 20,000 to 40,000 Units/24 hours in 1,000 mL of 0.9% Sodium Chloride Injection USP (or in any compatible solution for infusion.) As one of skill in the art would know, the dosage of heparin should be adjusted according to the patient's coagulation test results. For example, when heparin sodium is given by continuous intravenous infusion, the coagulation time should be determined approximately every four hours in the early stages of treatment. When the drug is administered intermittently by intravenous injection, coagulation tests should be performed before each injection during the early stages of treatment and at appropriate intervals thereafter. Dosage may be considered adequate when the activated partial thromboplastin time (APTT) is 1.5 to 2 times normal or when the whole blood clotting time is elevated approximately 2.5 to 3 times the control value. After deep subcutaneous (intrafat) injections tests for adequacy of dosage are best performed on samples drawn four to six hours after the injections.

Other than in the examples, and where otherwise indicated, all numbers used in the specification and claims are to be understood as modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following example is intended to illustrate the invention in a non-limiting manner.

### EXAMPLE 1

A study is performed to determine the compatibility of tigecycline solutions with heparin. Heparin is prepared for this study as per the directions given in the package insert. The compatibility testing is performed using a “simulated Y-site” technique developed and published by Trissel et. al. “Compatibility of Fenoldopam Mesylate with other Drugs during Simulated Y-site Administration, Am. J. Health-System Pharmacy, Vol. 60, 80-85, 2003; Trissel, et. al. “Physical Compatibility of Antithymocyte Globulin (Rabbit) with Heparin Sodium and Hydrocortisone Sodium, Vol. 60, 1650-1652, 2003; Trissel et. al. “Compatibility Screening of Bivalirudin during Simulated Y-site Administration with Other Drugs, and Trissel et. al. “Compatibility of piperacillin sodium plus tazobactum with selected drugs during simulated Y-site injection, Am. J. Hosp. Pharm., Vol. 51, 672-678, 1994.

To “simulate” the Y-site, solutions of both tigecycline and heparin are mixed in a 1:1 ratio, and the compatibility of the common solution is evaluated for Appearance and Description (i.e. color, visible particulates or turbidity), subvisible particulates by light obscuration (i.e. HIAC), and pH over a four hour, room temperature period. This time period has become standard when performing these “simulated Y-site” studies, although the actual residence time for the two solutions in the administration set may not exceed eight minutes prior to entering the venous system. The tigecycline test solution, before and after mixing with the counter test solution, is protected from atmospheric oxygen using a nitrogen overlay which mimics the actual “closed” system of an administration set.

The acceptance criteria of “no incompatibility indicating change” over the four hour evaluation period is being applied to the Appearance and Description assay as well as the HIAC subvisible particulate assay. Thus, for instance, formation of visible particulates or increase of subvisible particulates would indicate an incompatibility and would result in a “Y-site incompatible” classification for that diluent or drug. In addition to the Appearance and Description and HIAC testing, the pH of the solutions are monitored “for information only.” Before mixing of the two test solutions together; Appearance and Description, HIAC subvisible particulate testing, and pH are determined on each as controls.

Preparation of the Test Solutions

Preparation of Tigecycline Solution, 1 mg/ mL, in Normal Saline (“NS”.) For reconstitution and admixing of the tigecycline, 0.9% Sodium Chloride Injection, USP, 100 ml, is used.
1. Two×5.3 mL of NS is withdrawn from a 100 mL IV solution bag.
2. Two×tigecycline 50 mg vials are each reconstituted with 5.3 ml of NS. The vials are mixed with gentle shaking, avoiding excessive or vigorous shaking of the vial.
3. Two×5.0 ml of reconstituted tigecycline is transferred to the 100 mL NS IV solution bag used for the reconstitution. The solution is gently mixed to obtain homogeneity.
4. One hundred (100) ml of the 1 mg/ml tigecycline solution is transferred to a 250 ml beaker, flushed with nitrogen, and covered with Parafilm®

Preparation of Heparin
1. To prepare heparin in the usual dosage of 10 unit bolus in 1 ml, 10 ml are transferred from 10 vials of 10 units/ml to a clean beaker to result in 10 units/ml.
2. To prepare heparin in the usual dosage of 0.75-2 units/hour continuously, 10 ml of solution is removed from a 100 ml bag of 0.9% Sodium Chloride Injection, USP. 10 ml of heparin solution is transferred from one 1,000 units/ml×10 ml vial to the volume depleted bag of NS to result in 100 units/ml.

Testing of Controls
1. Approximately 30 ml of the tigecycline and of the heparin solution are transferred to separate beakers and evaluated for Appearance and Description, subvisible particulates by HIAC and pH testing before the two test articles are mixed together.
2. The remaining volumes of the two test articles are used immediately.

Preparation and Testing of Combined Solutions
1. Seventy (70) ml of the heparin solution is transferred to the beaker containing the approximate 70 ml of the tigecycline solution, combined, and mixed creating the initial sample. Approximately 30 ml of the combined solution is immediately removed for 0 hour Appearance and Description, subvisible particulates by HIAC and pH testing. The beaker with the remaining solution is then recovered with Parafilm and the headspace flushed with nitrogen.
2. The sampling and testing of 30 ml aliquots is repeated after one and four hours.

Testing Performed

The following tests are performed immediately after sampling:
1. Appearance and Description for color, visible particulates, and turbidity measured using a Black and White background light box with a fluorescent lamp capable of producing intensity of illumination between 2000 and 3750 lux.
2. Sub visible particulates are measured by light obscuration (HIAC) as per United States Pharmacopeia USP Chapter 788 Particulate Matter in Injections.

pH is measured using a potentiometric meter capable of measuring pH values reproducibly within 0.02 pH units and having electrodes suitable for pH meter use.

TABLE 1
Test Results for “Simulated Y-site” Compatibility Study of
Tigecycline, 1 mg/mL, in NS and Heparin, 10 Units/ml, in NS

| | | | | | Time after mixing, room temperature, ambient | | |
|---|---|---|---|---|---|---|---|
| | | Acceptance | | Before mixing | light, nitrogen flush of test sample head space | | |
| Assay | Units | Criteria | | 0 Hours | 0 Hours | 1 Hour | 4 Hours |
| Appearance | — | No | Tigecycline | Clear, yellow | Clear, | Clear, | Clear, yellow |
| and | | incompatibility | | solution, | yellow | yellow | solution, essentially |
| Description | | indicating | | essentially free | solution, | solution, | free from visible |
| | | change | | from visible | essentially | essentially | particulate |
| | | | | particulates | free from | free from | matter |
| | | | Heparin | Clear, colorless | visible | visible | |
| | | | | solution, | particulate | particulate | |
| | | | | essentially free | matter | matter | |
| | | | | from visible | | | |
| | | | | particulates | | | |
| HIAC | ≧10 μm | No | Tigecycline | 39.3 | 29.9 | 22.2 | 12.8 |
| | particles/mL | incompatibility | | 1.4 | 0.6 | 0.4 | 0.2 |
| | ≧25 μm | indicating | Heparin | 12.5 | | | |
| | particles/mL | change | | 0.6 | | | |
| pH | — | For information | Tigecycline | 7.78 | 7.78 | 7.74 | 7.80 |
| | | only | Heparin | 6.59 | | | |

TABLE 2
Test Results for “Simulated Y-site” Compatibility Study of
Tigecycline, 1 mg/mL, in NS and Heparin, 100 Units/ml, in NS

| | | | | | Time after mixing, room temperature, | | |
|---|---|---|---|---|---|---|---|
| | | | | | ambient light, nitrogen flush of test | | |
| | | | | Before mixing | sample head space | | |
| Assay | Units | Acceptance Criteria | | 0 Hours | 0 Hours | 1 Hour | 4 Hours |
| Appearance | — | No incompatibility | Tigecycline | Clear, yellow | Clear, | Clear, | Clear, |
| and Description | | indicating change | | solution, | yellow | yellow | yellow |
| | | | | essentially free | solution, | solution, | solution, |
| | | | | from visible | essentially | essentially | essentially |
| | | | | particulates | free from | free from | free from |
| | | | Heparin | Clear, colorless | visible | visible | visible |
| | | | | solution, | particulate | particulate | particulate |
| | | | | essentially free | matter | matter | matter |
| | | | | from visible | | | |
| | | | | particulates | | | |
| HIAC | ≧10 μm | No incompatibility | Tigecycline | 69.1 | 27.0 | 35.5 | 17.7 |
| | particles/mL | indicating change | | 6.2 | 0.7 | 1.5 | 0.4 |
| | ≧25 μm | | Heparin | 30.3 | | | |
| | particles/mL | | | 1.7 | | | |
| pH | — | For information | Tigecycline | 7.82 | 7.76 | 7.74 | 7.71 |
| | | only | Heparin | 5.79 | | | |

### EXAMPLE 2

A study is performed to determine the compatibility of tigecycline solutions with various other diluents and drugs.

All drug products are prepared per package insert directions; some drugs having wide therapeutic ranges are tested at multiple concentrations, and some drugs with different formulations are tested. A simulation of the mixing which would occur at an intravenous administration set Y-site is used, and the period for evaluation of compatibility exaggerated the generally short time that the two agents would be together prior to venous circulation. Test solutions before and after mixing are measured by visual observation, by light obscuration for subvisible particulates (HIAC), and by potentiometry over a four hour period.

The acceptance criteria of “no incompatibility indicating change” is used in assessing the data. The results are categorized into the following four classifications, as influenced by package insert statements:
1. “Compatible”, i.e. mixtures of tigecycline with diluents or other drug show no incompatibility indicating changes.
2. “Compatible with manufacture's restrictions”, i.e. mixtures of tigecycline with drug B show no incompatibility indicating changes, but the manufacturer of drug B indicates that Y-site administration should not be performed with their drug.
3. “Incompatible”, i.e. mixtures of tigecycline with diluents or other drug show incompatibility indicating changes.
4. “Incompatible with manufacture's restrictions”, i.e. mixtures of tigecycline with other drugs show incompatibility indicating changes, and the manufacturer of other drugs indicates that Y-site administration should not be performed with their drug.

The compatibility results are listed in Table 3 and are summarized as follows:

TABLE 3
Compatibility Classifications and Concentrations of Intravenous Diluents and Drugs
Tested under Simulated Y-site Conditions with Tigecycline for Injection

| | | Compounding Directions | | |
|---|---|---|---|---|
| | | as Per Manufacture's | Compatibility | |
| Diluent | Usual Dose | Package Insert | Classification | Comments |
| 5% Dextrose Injection | Continuous IV | Used as is | Compatible | none |
| 5% Dextrose and 0.9% | Continuous IV | Used as is | Compatible | none |
| Sodium Chloride Injection | | | | |
| 5% Dextrose in Lactated | Continuous IV | Used as is | Compatible | none |
| Ringers Injection | | | | |
| 5% Dextrose, 0.45% | Continuous IV | Used as is | Compatible | none |
| Sodium Chloride and 0.15% | | | | |
| Potassium Chloride | | | | |
| Injection | | | | |
| Lactated Ringers Injection | Continuous IV | Used as is | Compatible | none |
| Plasma-Lyte 56/5% | Continuous IV | Used as is | Compatible | none |
| Dextrose Injection | | | | |

Intravenous Drugs Frequently Administered with Tigecycline by Continuous Infusion

| | | Compounding Directions as Per | Final | | Compatibility | |
|---|---|---|---|---|---|---|
| Drug | Usual Dose | Manufacture's Package Insert | Concentration | | Classification | Comments |
| Amikacin | 1 g over 30 | Removed 2 ml each from 5 vials of 100 mg/ | 5 | mg/ml | Compatible | No comments |
| | minutes | 2 ml Amikacin and added to the | | | | |
| | | volume depleted 100 ml NS container | | | | |
| Dobutamine | 2.5 mcg/kg/ | Removed 1.6 ml from a 250 mg/20 ml vial | 0.2 | mg/ml | Compatible | No comments |
| | minute | and diluted with 98.4 ml NS to result in | | | | |
| | continuously | 0.2 mg/ml | | | | |
| | 10 mcg/kg/ | Removed 8.0 ml from a 250 mg/20 ml vial | 1.0 | mg/ml | Compatible | No comments |
| | minute | and diluted with 92 ml NS to result in 1.0 mg/ml | | | | |
| | continuously | | | | | |
| Dopamine | 0.5-3 mcg/kg/ | Transferred 4 ml of 40 mg/ml to a 100 ml | 1.6 | mg/ml | Compatible | No comments |
| | minute | NS bag to result in 1.6 mg/ml | | | | |
| | continuously | | | | | |
| Epinephrine | 1 to 10 μg/min | Transferred 0.4 ml each from one | 4 | mcg/ml | Compatible | No comments |
| | | 1 ml × 1 mg/ml ampoule and ad to a 100 ml | | | | |
| | | of NS to result in 4 mcg/ml | | | | |
| Gentamycin | 350 mg over 30 | Removed 50 ml each from 2 × 50 ml bags | 1.4 | mg/ml | Compatible | No comments |
| | minutes | of 1.4 mg/ml Gentamycin and used as is | | | | |
| Lidocaine | 1-2 mg/minute | Transferred 20 ml from each of five 20 ml | 2% | | Compatible | No comments |
| | continuously | vials of 2% lidocaine to a clean beaker to | | | | |
| | | result in 2% lidocaine. | | | | |
| Potassium | 20-40 mEq/L | Removed 15 ml NS from a 100 ml bag. | 0.3 | mEq/L | Compatible | No comments |
| (as | continuously | Transfer 15 ml of 2 mEq/ml Potassium | | | | |
| potassium | | chloride from one 20 ml vial to the NS bag | | | | |
| chloride) | | to result in 0.3 mEq/L. | | | | |
| Ranitidine | 6.5 mg/hour | Transferred 2.4 ml of 25 mg/ml, 2 ml | 0.6 | mg/ml | Compatible | No comments |
| | continuously | vials, from two vials to 100 ml of NS to | | | | |
| | | result in 0.6 mg/ml. | | | | |
| Theophyline | 0.4 to 4 mg/ml, | Used as is, as 1.6 mg/ml in D5W. | 1.6 | mg/ml | Compatible | No comments |
| | not to exceed | | | | | |
| | 20 mg/min | | | | | |
| Tobramycin | 350 mg over 30 | Removed 2 ml each from 12.5 vials of 20 mg/ | 2.5 | mg/ml | Compatible | No comments |
| | minutes | 2 ml Tobramycin and added to the | | | | |
| | | volume depleted 100 ml NS container | | | | |

Intravenous Drugs Potentially Administered with Tigecycline by Intermittent Infusion

| | | Compounding Directions as Per | Final | | Compatibility | |
|---|---|---|---|---|---|---|
| Drug | Usual Dose | Manufacture's Package Insert | Concentration | | Classification | Comments |
| Amphotericin B | 2 mg/mL over | Two 50 mg vials are reconstituted as | 2 | mg/mL | Incompatible* | Cloudy solution formed |
| for Injection | 120 minutes | follows: “An initial concentrate of 5 mg | | | | immediately and HIAC |
| | | per ml is first prepared by rapidly | | | | particulates formed |
| | | expressing 10 ml SWFI directly into the | | | | within 1 hour |
| | | lyophilized cake, using a sterile needle | | | | |
| | | (minimum diameter 20 gauge) and | | | | |
| | | syringe. Shake the vial immediately | | | | |
| | | until the colloidal solution is clear.” The | | | | |
| | | infusion solution is then obtained by | | | | |
| | | transferring the two 10 ml reconstituted | | | | |
| | | volumes to the 80 ml of 5% Dextrose | | | | |
| | | Injection, USP to result in 2 mg/ml. | | | | |
| Amphotericin B | 2 mg/mL over | Performed simulated Y-site testing | 2 | mg/mL | Incompatible | By nature of the lipid |
| Lipid Complex | 120 minutes | despite the following PI statement: | | | with | complex of this |
| Injection | | “DO NOT DILUTE WITH SALINE | | | manufacturer's | formation resulting in |
| | | SOLUTIONS OR MIX WITH | | | restrictions | high HIAC counts, |
| | | OTHER DRUGS OR | | | | sub visible particulate |
| | | ELECTROLYTES as the | | | | matter formation |
| | | compatibility of ABELCET with | | | | cannot be excluded |
| | | these materials has not been | | | | and therefore an |
| | | established. An existing | | | | incompatible |
| | | intravenous line should be flushed | | | | classification is |
| | | with 5% Dextrose Injection before | | | | appropriate |
| | | infusion of ABELCET, or a separate | | | | |
| | | infusion line should be used.” | | | | |
| | | Diluted 20 ml of 5 mg/ml with 80 ml | | | | |
| | | of D5W to result in 2 mg/ml. | | | | |
| Azithromycin | 500 mg over 60 | “Zithromax is supplied under | 2 | mg/ml | Compatible | No comments |
| | minutes | vacuum. It is recommended that a | | | | |
| | | standard 5 ml (non-automated | | | | |
| | | syringe be used to ensure that the | | | | |
| | | exact amount of 4.8 ml of SWFI is | | | | |
| | | dispensed.” Added 4.8 ml of SWFI | | | | |
| | | to the 500 mg vial and shake until all | | | | |
| | | the drug is dissolved. Transfer 2 ml | | | | |
| | | of the reconstituted solution to a 100 ml | | | | |
| | | bag of NS to result in 2 mg/ml. | | | | |
| Aztreonam | 20 mg/mL | Reconstituted two 1 gram vials with | 20 | mg/ml | Compatible | No comments |
| | | “at least 3 ml of SWFI” and transfer | | | | |
| | | contents to a 100 ml bag of NS. | | | | |
| Cefepime | 2 g/50 mL over | Removed four × 10 ml volumes from | 40 | mg/ml | Compatible | No comments |
| | 20 minutes | a 100 ml bag NS. Add 10 ml to | | | | |
| | | each of four 1 g vials, and transfer | | | | |
| | | contents (11.3 ml) to volume | | | | |
| | | depleted NS bag to result in 40 mg/ml | | | | |
| | | in NS. | | | | |
| Cefotaxime | 2 g/50 mL over | Removed four × 10 ml volumes from | 40 | mg/ml | Compatible | No comments |
| | 20 minutes | a 100 ml bag of NS. Add 10 ml to | | | | |
| | | each of four 1 g vials, shake to | | | | |
| | | dissolve. Transfer 10.4 ml from | | | | |
| | | each of the four reconstituted vials | | | | |
| | | to the volume depleted bag of NS to | | | | |
| | | result in 40 mg/ml in NS. | | | | |
| Ceftazidime | 2 g/50 mL over | Removed four 10 ml volumes from a | 40 | mg/ml | Compatible | No comments |
| | 20 minutes | 100 ml bag NS. Add 10 ml to each | | | | |
| | | of four 1 g vials, and transfer | | | | |
| | | contents to volume depleted NS bag | | | | |
| | | to result in 40 mg/ml in NS. | | | | |
| Ceftriaxone | 2 g/50 mL over | Removed four × 9.6 ml volumes | 40 | mg/ml | Compatible | No comments |
| | 20 minutes | from a 100 ml bag NS. Add 9.6 ml | | | | |
| | | to each of four 1 g vials, and | | | | |
| | | transfer contents to volume depleted | | | | |
| | | NS bag to give 40 mg/ml in NS. | | | | |
| Chlorpromazine | 25-50 mg | Removed 1.0 ml from each of four × 25 mg/ | 1 | mg/ml | Incompatible* | Visual particulates |
| | (1 mg/mL) over | 1 ml ampoules and dilute | | | | formed immediately |
| | 25-50 minutes | with 96 ml of NS to result in 1 mg/ml. | | | and HIAC | |
| | | | | | | particulates after 1 |
| | | | | | | hour |
| Cimetidine | 300 mg/50 mL | Removed 2.0 ml from each of two × 300 mg/ | 6 | mg/ml | Compatible | No comments |
| | over 20 minutes | 2 ml vials and dilute with 96 ml | | | | |
| | | of NS to result in 6 mg/ml. | | | | |
| Ciprofloxacin | 1 mg/mL | Performed simulated Y-site testing | 1 | mg/ml | Compatible with | No comments |
| | | despite the following PI statement: | | | manufacturer's | |
| | | “If the Y-type or “piggyback” method | | | restrictions | |
| | | of administration is used, it is | | | | |
| | | advisable to discontinue temporarily | | | | |
| | | the administration of any other | | | | |
| | | solutions during the infusion of | | | | |
| | | Ciprofloxacin (CIPRO) IV If the | | | | |
| | | concomitant use of CIPRO IV and | | | | |
| | | another drug is necessary each | | | | |
| | | drug should be given separately in | | | | |
| | | accordance with the recommended | | | | |
| | | dosage and route of administration | | | | |
| | | for each drug..” Removed 10 ml | | | | |
| | | from one vial of 400 mg/40 ml and | | | | |
| | | dilute with 90 ml of NS to result in 1 mg/ml. | | | | |
| Etrapenem | 1 g/50 mL | Performed simulated Y-site testing | 20 | mg/ml | Compatible with | No comments |
| | | despite the following PI statement: | | | manufacturer's | |
| | | “DO NOT MIX OR CO-INFUSE | | | restrictions | |
| | | INVANZ WITH OTHER | | | | |
| | | MEDICATIONS.” Reconstituted two | | | | |
| | | 1 g vials with 10 ml of NS, shake | | | | |
| | | well to dissolve, and immediately | | | | |
| | | transfer to approximately 80 ml of | | | | |
| | | NS to result in 20 mg/ml. | | | | |
| Fluconazole | 400 mg (2 mg/ml) | Used “as is” as a 2 mg/ml solution. | 2 | mg/ml | Compatible | No comments |
| | over | | | | | |
| | 120 minutes | | | | | |
| Haloperidol | 0.2 mg/mL | Transferred 1 ml from four 1 ml vials | 0.2 | mg/ml | Compatible | No comments |
| | | of 5 mg/ml to a 100 ml bag of NS to | | | | |
| | | result in 0.2 mg/ml. | | | | |
| Imipenem/ | 500 mg/100 mL | Removed 40 ml NS from a 100 ml | 5 | mg/ml | Compatible | No comments |
| cilastatin | over 15-30 | bag of NS. Add 10 ml of NS to each | | | | |
| | minutes | of two 250 mg vials, “shake well and | | | | |
| | | transfer the resulting suspension to | | | | |
| | | the infusion solution container.” | | | | |
| | | “Repeat with an additional 10 ml of | | | | |
| | | infusion solution to ensure complete | | | | |
| | | transfer of vial contents to the | | | | |
| | | infusion solution. The resulting | | | | |
| | | mixture should be agitated until | | | | |
| | | clear.” The resulting solution is 5 mg/ml. | | | | |
| Linezolid | 600 mg/300 mL | Performed simulated Y-site testing | 2 | mg/ml | Compatible with | No comments |
| | over 30-120 | despite the following PI statement: | | | manufacturer's | |
| | minutes | “Do not use this intravenous infusion | | | restrictions | |
| | | bag in series connections. If Zyvox | | | | |
| | | IV is to be given concomitantly with | | | | |
| | | another drug, each drug should be | | | | |
| | | given separately in accordance with | | | | |
| | | the recommended dosage and route | | | | |
| | | of administration.” Use as is as | | | | |
| | | 2 mg/ml. | | | | |
| Methylprednisolone | 30 mg/kg/50 mL | Removed approximately 32 ml from | 20 | mg/ml | Incompatible* | By the nature of this |
| | over 30 minutes | a 100 ml bag of NS. “Activate” 16 | | | | formulation resulting |
| | | “ACT-O-VIALS” by pressing down | | | | in high HIAC counts, |
| | | on plastic activators. Gently agitate | | | | sub visible particulate |
| | | to effect solution. Remove plastic | | | | matter formation |
| | | tab., invert, and transfer 2 ml from | | | | cannot be excluded |
| | | each of the 16 vials to the volume | | | | and therefore an |
| | | depleted bag to result in 20 mg/ml. | | | | incompatible |
| | | | | | | classification is |
| | | | | | | appropriate |
| Metocloopramide | 5 mg/mL | Removed two ml each from 50 vials | 5 | mg/ml | Compatible | No comments |
| | | of 10 mg/2 ml metoclopramide and | | | | |
| | | transfer to a clean 100 ml beaker for | | | | |
| | | the 5 mg/ml solution. | | | | |
| Piperacillin/ | 3/0.375 g q6h | Removed 15 ml of NS from a 100 ml | 3.375 | mg/ml | Compatible | No comments |
| tazobactam | | bag and reconstitute one 3.375 g | | | | |
| | | vial of Piperacillin/tazobactam | | | | |
| | | (Zosyn). Transfer the reconstituted | | | | |
| | | Zosyn to the volume depleted bag of | | | | |
| | | NS to result in 33.75 mg/ml. | | | | |
| Vancomycin | 1 g/200 mL over | Reconstituted by adding 10 ml of | 5 | mg/ml | Compatible | No comments |
| | 60 minutes | SWFI to a 500 mg vial, and dilute | | | | |
| | | with 100 ml NS to result in 5 mg/ml. | | | | |
| | | “Vancomycin solution has a low pH | | | | |
| | | and may cause physical instability of | | | | |
| | | other compounds.” | | | | |
| Voriconazole | 3-6 mg/kg | Performed simulated Y-site testing | 2 | mg/ml | Incompatible | By the nature of this |
| | (2 mg/ml) over | despite the following PI statement: “ | | | with | formulation resulting |
| | 120 minutes | VFEND IV must not be infused into | | | manufacturer's | in high HIAC counts, |
| | | the same line or canula | | | restrictions | sub visible particulate |
| | | concomitantly with other drug | | | | matter formation |
| | | infusions, including parenteral | | | | cannot be excluded |
| | | nutrition. Reconstituted one 200 mg | | | | and therefore an |
| | | vial with 19 ml NS, shook until all of | | | | incompatible |
| | | the powder is dissolved, and added | | | | classification is |
| | | to the volume depleted 100 ml NS | | | | appropriate. |
| | | container to result in a 2 mg/ml. | | | | |

*Test sample is not protected from light, higher concentration are used
*Test sample is not protected from light.

TABLE 4
Example of Tigecycline - higher pH solution plus Dobutamine - lower pH solution, resulting in higher pH
(after mixing) without change in Appearance and Description and Subvisible Particulates counts

| | | Acceptance | | Before Mixing | After Mixing | | |
|---|---|---|---|---|---|---|---|
| Assay | Units | Criteria | Components | 0 hours | 0 hours | 1 hour | 4 hours |
| Appearance | — | No incompatibility | Tigecycline | Clear, yellow solution | Clear, yellow | Clear, yellow | Clear, yellow |
| and | | indicating change | | essentially free from | solution | solution | solution |
| Description | | | | visible particulates | essentially | essentially free | essentially free |
| | | | Dobutamine | Clear, colorless | free from | from visible | from visible |
| | | | | solution essentially | visible | particulates | particulates |
| | | | | free form visible | particulates | | |
| | | | | particulates | | | |
| Subvisible | ≧10 μm | No incompatibility | Tigecycline | 35.8 | 22.2 | 13.4 | 26.2 |
| Particulates | particles/ml | indicating change | | 0.9 | 0.8 | 0.2 | 0.4 |
| (HIAC) | ≧25 μm | | Dobutamine | 5.9 | | | |
| | particles/ml | | | 0.6 | | | |
| pH | — | For | Tigecycline | 7.81 | 7.52 | 7.63 | 7.59 |
| | | information only | Dobutamine | 4.18 | | | |

TABLE 5
Example of Tigecycline - higher pH solution plus Dopamine - lower pH solution, resulting in lower pH
(after mixing) without change in Appearance and Description and Subvisible Particulates counts

| | | Acceptance | | Before Mixing | After Mixing | | |
|---|---|---|---|---|---|---|---|
| Assay | Units | Criteria | Components | 0 hours | 0 hours | 1 hour | 4 hours |
| Appearance | — | No | Tigecycline | Clear, yellow solution | Clear, yellow | Clear, yellow | Clear, yellow |
| and | | incompatibility | | essentially free from | solution | solution | solution |
| Description | | indicating | | visible particulates | essentially free | essentially free | essentially free |
| | | change | Dopamine | Clear, colorless | from visible | from visible | from visible |
| | | | | solution essentially | particulates | particulates | particulates |
| | | | | free from visible | | | |
| | | | | particulates | | | |
| Subvisible | ≧10 μm | No | Tigecyline | 82.3 | 37.4 | 25.5 | 13.3 |
| Particulates | particles/ml | incompatibility | | 2.2 | 0.6 | 0.1 | 0.4 |
| (HIAC) | ≧25 μm | indicating | Dopamine | 3.1 | | | |
| | particles/ml | change | | 0.1 | | | |
| pH | — | For | Tigecycline | 7.84 | 4.69 | 4.76 | 4.72 |
| | | information only | Dopamine | 3.59 | | | |

TABLE 6
Example of Tigecycline - higher pH solution plus Chlorpromazine - lower pH solution, resulting in higher pH
(after mixing) with change in Appearance and Description and Subvisible Particulates counts

| | | Acceptance | | Before Mixing | After Mixing | | |
|---|---|---|---|---|---|---|---|
| Assay | Units | Criteria | Components | 0 hours | 0 hours | 1 hour | 4 hours |
| Appearance | — | No | Tigecycline | Clear, yellow | Hazy, | Turbid, yellow | Turbid, yellow |
| and | | incompatibility | | solution essentially | yellow | suspension | suspension |
| Description | | indicating | | free from visible | suspension | | |
| | | change | | particulates | | | |
| | | | Choloropromazine | Clear, colorless | | | |
| | | | | solution essentially | | | |
| | | | | free from visible | | | |
| | | | | particulates | | | |
| Subvisible | ≧10 μm | No | Tigecycline | 27.4 | 42.5 | 7465.3 | 17070.0 |
| Particulates | particles/ml | incompatibility | | 1.3 | 1.2 | 1.3 | 0.2 |
| (HIAC) | ≧25 μm | indicating | Chloropromazine | 21.9 | | | |
| | particles/ml | change | | 2.9 | | | |
| pH | — | For | Tigecycline | 7.74 | 7.01 | 7.10 | 7.15 |
| | | information only | Chloropromazine | 4.61 | | | |

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

What is claimed is:

1. A composition comprising at least one glycylcycline chosen from a compound of formula I 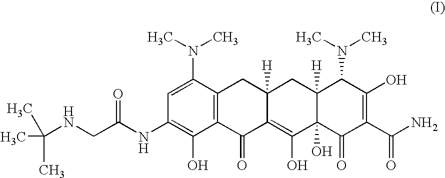
and pharmaceutically acceptable salts thereof, and at least one heparin

2. A composition according to claim 1, wherein the glycylcycline is a free base.

3. A composition according to claim 1, wherein the heparin is heparin sodium.

4. A composition according to claim 1, wherein the composition is suitable for parenteral administration.

5. A composition according to claim 1, wherein the composition is suitable for intravenous administration.

6. A pharmaceutical composition as claimed in claim 1, further comprising
at least one pharmaceutically acceptable excipient.

7. A combination therapy comprising administration of at least one glycylcycline chosen from a compound of formula I 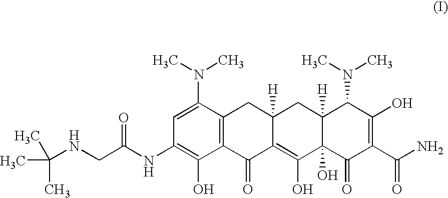
and pharmaceutically acceptable salts thereof, and at least one heparin, wherein administration is simultaneous, separate or sequential.

8. A combination therapy according to claim 7, wherein there is no incompatibility as determined by at least one test chosen from color change, gas formation, visible particulate formation, sub-visible particle formation and turbidity.

9. A combination therapy according to claim 7, wherein the glycylcycline is administered between two hours before and two hours after the heparin is administered.

10. A medical apparatus comprising at least two separate compartments, wherein a first compartment comprises at least one glycylcycline chosen from a compound of formula I 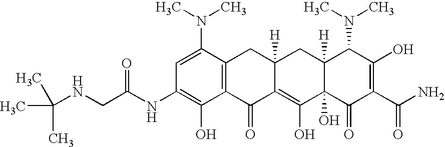
(I)
and pharmaceutically acceptable salts thereof and a second compartment comprising at least one heparin, and wherein the first and second compartments are connected to at least one administration set.

11. A medical apparatus according to claim 10, wherein the first and second compartments are connected to the same administration set and are mixed prior to administration.

12. A medical apparatus according to claim 11, wherein the first and second compartments are connected to the same administration set and are mixed at a Y-site prior to administration.

13. A medical apparatus according to claim 10, wherein flushing of an administration set is not required.

14. A method for administering at least one glycylcycline and at least one heparin, comprising administering to a patient in need thereof a therapeutically effective amount of the at least one glycylcycline chosen from a compound of formula I 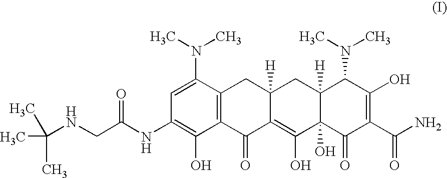
and its pharmaceutically acceptable salts, and administering to a patient in need thereof a therapeutically effective amount of the at least one heparin.

15. A method for administering glycylcycline and heparin, comprising administering to a patient in need thereof a therapeutically effective amount of at least one glycylcycline chosen from a compound of formula I 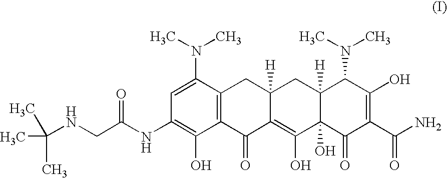
and its pharmaceutically acceptable salts, and administering to a patient in need thereof a therapeutically effective amount of at least one heparin, wherein the glycylcycline and the heparin are administered through the same site of administration.

16. A method of treating complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) comprising administering to a patient in need thereof a therapeutically effective amount of at least one glycylcycline chosen from a compound of formula I 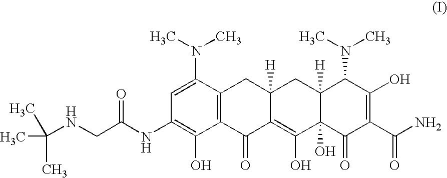
and its pharmaceutically acceptable salts, and administering to a patient in need thereof a therapeutically effective amount of at least one heparin.

17. A method of treating complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof, which comprises providing to said patient an effective amount of a combination comprising a glycylcycline of formula I or a pharmaceutically acceptable salt thereof and a heparin or a pharmaceutically acceptable salt thereof.

18. A method of administering an antibiotic comprising administering to a patient in need thereof a therapeutically effective amount of at least one glycylcycline chosen from a compound of formula I 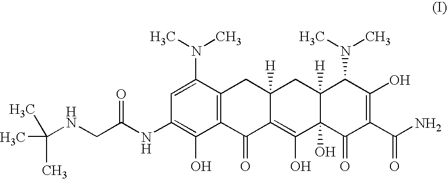
and its pharmaceutically acceptable salts, and administering to a patient in need thereof a therapeutically effective amount of at least one heparin.

19. An article of manufacture, comprising (a) a dosage form at least one glycylcycline chosen from a compound of formula I 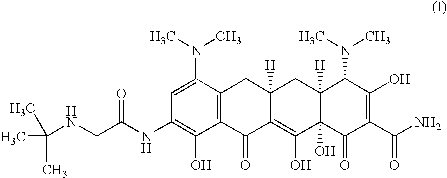
and its pharmaceutically acceptable salts,
(b) a package insert or printed labeling providing that a dosage form of the glycylcycline may be co-administered with heparin without flushing of a common administration site; and
(c) at least one compartment in which the glycylcycline is stored.

20. A kit comprising: (a) at least one dosage form of at least one glycylcycline chosen from a compound of formula I 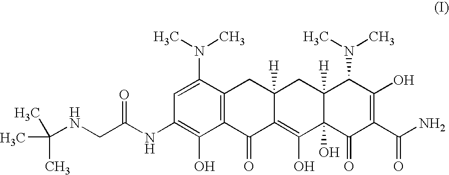
and its pharmaceutically acceptable salts; (b) at least one compartment in which the glycylcycline is stored; and (c) a package insert comprising: i) information regarding the dosage amount and duration of exposure of a dosage form of the glycylcycline and ii) providing that the dosage form of the glycylcycline may be administered with Heparin at a common point of administration.

21. Use of a glycylcycline of formula I or a pharmaceutically acceptable salt thereof in the preparation of a medicament for the treatment of complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof, which treatment also comprises administration of a heparin of a pharmaceutical salt thereof.

22. Use of a heparin or a pharmaceutically acceptable salt thereof in the preparation of a medicament for the treatment of complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof, which treatment also comprises administration of a glycylcycline of formula I or a pharmaceutical salt thereof.

23. A product comprising a glycylcycline of formula I or a pharmaceutically acceptable salt thereof and a heparin or a pharmaceutically acceptable salt thereof as a combined preparation for simultaneous, separate or sequential use in the treatment of complicated intra-abdominal infections (cIAI) and complicated skin and skin structure infections (cSSSI), caused by gram-negative and gram-positive pathogens, anaerobes, and both methicillin-susceptible and methicillin-resistant strains of Staphylococcus aureus (MSSA and MRSA) in a patient in need thereof.
